(19) United States
(12) Patent Application Publication
(10) Pub. No.: US 2009/0142428 A1
(43) Pub. Date: Jun. 4, 2009

(54) Extracts from the Bark of Corynanthe Species and Use Thereof as Well as Medicaments, Dietetic Food Products and Pharmaceutical Preparations Containing Said Extracts

(75) Inventors: Egon Koch, Karlsruhe (DE); Hermann Hauer, Karlsruhe (DE)

Correspondence Address: EDWARDS ANGELL PALMER & DODGE LLP, P.O. BOX 55874, BOSTON, MA, 02205, US

(73) Assignee: DR. WILLMAR SCHWABE GMBH & CO. KG, Karlsruhe (DE)

(21) Appl. No.: 12/084,745

(22) PCT Filed: Nov. 7, 2006
(86) PCT No.: PCT/EP2006/010663

§ 371 Date: May 8, 2008

(30) Foreign Application Priority Data
Nov. 8, 2005 (DE) 10 2005 053 241.1

(51) Int. Cl. A61K 36/00; A61P 29/00; A61P 3/00

(52) U.S. Cl. 424/775

(57) ABSTRACT

The present invention relates to extracts from the bark of corynanthe species, in particular corynanthe pachyceras as well as to their use for the therapy and prophylaxis of diseases of the lower urinary tract, sexual disorders, lipid metabolism disorders, cardiovascular diseases and acute and chronic pain conditions. The present invention further relates to medicaments, dietetic food products and pharmaceutical preparations containing these extracts.

The present invention relates to extracts from the bark of corynanthe species, in particular corynanthe pachyceras, as well as their use for the therapy and prophylaxis of diseases of the lower urinary tract, sexual dysfunctions, disorders of the lipid metabolism, cardiovascular diseases and acute and chronic pain conditions. The present invention further relates to medicaments, dietetic food products and pharmaceutical preparations containing said extracts.

Benign prostatic hyperplasia (BPH) and the accompanying lower urinary tract symptoms (LUTS) are by far the most important urologic diseases in male humans. Estimations assume that one third of the male humans above age 50 develop LUTS in the course of their life and that a surgical intervention will be necessary for 25% of them. The percentage of male humans suffering from BPH/LUTS increases to more than 90% until the 9^{th} decade of their life. In the Federal Republic of Germany about 4 million patients are treated against these complex of symptoms a year. In view of the growing expectancy of life and the increased health awareness, a further increase of the disease frequency has to be bargained for in the future (R. B. Moreland et al., J. Pharmacol. Exp. Ther. 2004, 308, 797).

Despite the large clinical importance of BPH/LUTS the aetiology and the pathogenesis of this disease have not been elucidated to a large extent. In addition to an increased age, an endogenous production of androgens is an important pre-condition for the development of BPH. In general, BPH is thus considered as an endocrinopathy of senescent men, which develops as a consequence of the hormonal reorganization with growing age.

Under a histologic point of view, BPH is the benign growth of the epithelial and stromal parts of the prostate. Due to the localization of the prostate at the urethra near the exit of the bladder, urinary obstructions which are accompanied by symptoms such as pollacisuria and nycturia as well as an incomplete and delayed micturition, occur due to an enlargement of the organ. In an advanced stage renal insufficiency and uraemia may occur as a result of the urinary stasis. In addition, due to stasis of secretions and urinary retentions, an abacterial prostatitis, congestions and recurring urinary tract infections develop, which are responsible for irritative micturition disorders in addition to obstructive complaints.

In addition to a static component which is due to an enlargement of the prostate and the mechanical micturition disorders caused thereby, a dynamic component appears to be involved in BPH/LUTS which is elucidated by an increased tonus of the smooth muscles. The extent of involvement of both mechanisms may vary largely from patient to patient. This explains that there is only a marginal correlation between the size of the prostate and the severity of the symptoms (C. G. Roehrborn and D. A. Schwinn, J. Urol. 2004, 171, 1029). Basically similar changes of the tonus of the smooth muscles are also involved in other diseases of the lower urinary tract, also in female humans, such as stress incontinence, urge incontinence and disorders of emptying the bladder.

Whereas the term BPH is reserved for the histologic or macroscopic diagnosis of hyperplasia of the prostatic gland, the accompanying LUTS such as urge to urinate, pollacisuria and nycturia as well as an incomplete and delayed micturition prevail for the patients. The variety of treatment alternatives for BPH ranges from an wait-and-see attitude (“watchful waiting”) to an open prostatectomy. Due to its effectiveness, the transurethral resection of the prostate, which about 33,500 male humans are subjected to per year in Germany, is considered to be the Golden Standard of surgical methods. However, the considerable risk of morbidity and mortality of invasive treatment methods is not acceptable for many patients, in particular patients with a lower severity of BPH, which explains the increasing importance of therapy approaches with medicaments. In addition to phytopharmaceuticals which are frequently used in case of less severe disease stages, the variety of medicaments includes primarily α₁-antagonists and 5-α-reductase inhibitors (E. Koch, Planta Med. 67, 489-500 (2001)).

The use of α₁-antagonists is based on the view that the dynamic component of BPH/LUTS is caused by an increased tonus of the smooth prostatic muscles which is mediated by an elevated release of noradrenaline from sympathic neurons. Today it is generally acknowledged that predominantly α_{1A}-adrenoceptors and α_{1D}-adrenoceptors are expressed in the prostate. The results of clinical studies show that α-receptor blockers produce a clinically significant improvement of the symptoms and of the maximum urinary flow. A particular advantage of α-receptor blockers is their rapid onset of action. However, there is currently no convincing evidence that they impede the further enlargement of the prostate. The side effects of α-receptor blockers include dizziness, headaches, weakness, or—thostatic dysregulation, rhinitis and sexual dysfunction (retrograde ejaculations) which are prevalently caused by the action of α-receptor blockers in the CNS and the cardiovascular system. The development of subtype specific α-receptor blockers which predominantly inhibit α_{1A}-receptors and α_{1D}-receptors (for example tamsulosin), intends to reduce the frequency of occurrence and the severity of side effects. However, the non-selective α₁-antagonist alfuzosin interestingly exhibits a similarly favourable profile of side effects as tamsulosin which is generally described as a uroselective α₁-receptor blocker. Besides the pharmacodynamic actions the pharmacokinetic-properties appear to significantly contribute to the uroselectivity. Hence, for example, effects on the blood pressure can be prevented by means of a slow dosage or sustained release formulations. Moreover, also α_{1A}-receptors are involved in the control of the blood pressure in addition to α_{1B}-receptors (R. B. Moreland et al., J. Pharmacol. Exp. Ther. 308, 797, 2004; C. G. Roehrborn and D. G. Schwinn, J. Urol. 171, 1029, 2004) which limits the possibilities to develop uroselective α-receptor blockers.

In general, a BPH develops in the presence of biologically effective male sex hormones only. A BPH has virtually never been observed in men which had to undergo castration before the age of 40 or in which no formation of androgens or an insufficient formation of androgens only occurs in the gonads due to a hypofunction of the hypophysis. Likewise, the normal development of the prostate and the development of a BPH does not occur in the case of a hereditary defect or in the absence of androgenic receptors (for example testicular feminization). The biologically most important androgen is dihydrotestosterone (DHT) which is formed locally from testosterone under the influence of 5-α-reductase. Since DHT pre-dominantly stimulates the epithelial components of BPH, a standstill of the growth or an atrophy of the glandular component can be achieved by inhibiting 5-α-reductase. However, the stromal component of BPH is virtually not effected at all. For example, the uptake of the 5-α-reductase inhibitor finasteride leads to a reduction of the prostatic DHT concentration of up to 85%, whereas the average reduction of the prostate size amounts to about 20% only and additionally requires a period of up to 12 months. This effect is of clinical relevance essentially only in cases where the volume of the prostate is higher than 40 ml at the beginning of the therapy.

Although androgens play a central role in the normal development and function of the prostate as well as in the development of a BPH, male sexual hormones alone are not sufficient to elicit the growth of prostatic cells. Numerous experimental investigations show that the growth stimulating effects of androgens in vivo are mediated by the local synthesis of growth factors and that dysfunctions in the paracrine and autocrine-mechanisms of the growth control within the epithelial and stromal components of the prostate are substantially involved in the development of PBH. A variety of growth factors (for example Epidermal Growth Factor [EGF], Transforming Growth Factor-α [TGF-α], TGF-β, basic Fibroblast Growth Factor (bFGF), Keratinocyte Growth Factor [KGF], Nerve Growth Factor [NGF], Insulin-like Growth Factor I [IGF-1] etc.) and their receptors have actually been detected in the prostate. Since BPH is very frequently accompanied by inflammatory reactions which presumably play an important role in the pathogenesis, Platelet-derived Growth Factor (PDGF), which is released for example by fibroblasts, thrombocytes and leucocytes, is probably of particular importance for the proliferation of prostatic cells (C. J. Vlahos et al., J. Cell. Biochem. 52, 404-413 (1993)). Growth factors mediate their biological action by binding to specific receptors at the surface of the cells, which have an intrinsic tyrosine kinase activity. After binding the ligand, the phosphorylation of the tyrosine residues occurs in the intracellular receptor domains, which subsequently elicits a cascade of intracellular reactions. The stimulation of the protein synthesis and DNA synthesis as well as the activation of the cell proliferation are among these reactions. Thus, inhibitors of receptor tyrosine kinase are considered to be promising substances for the development of new medicaments for the treatment of diseases which are accompanied by an increased cell proliferation (for example cancer, atherosclerosis, psoriasis) (A. Levitzki and A. Gazit, Science 267, 1782-1788 (1995)). However, little attention has been paid to this mode of action in the therapy of BPH up to now.

In recent years it has been demonstrated in different epidemiological investigations that there is a close correlation between BPH/LUTS and the occurrence of an erectile dysfunction (ED). For example, in the MSAM-7 study it has been demonstrated that the prevalence of EP in men without LUTS at an age between 50 and 80 years is about 25%. This rate increases in patients with severe symptoms to more than 80%. Furthermore, the frequency of occurrence of ED is increased by other simultaneously existing diseases such as hypertension, diabetes, hypercholesterolemia, angina pectoris and depressions (M. Shabbir et al., Curr. Med. Res. Opin. 20, 603, 2004).

The relaxation of the penis is predominantly maintained by the binding of noradrenaline to α_{1A}-receptors and α_{1B}-receptors in the corpus cavernosum. It is thus not surprising that an improvement in the sexual function was observed in patients with ED by therapy with α-receptor blockers (for example doxazosin and tamsulosin). In contrast, erections are predominantly mediated by the vasodilatory effect of nitric oxide (NO). NO is released from nitrergic neurons and is further synthesized by endothelial cells in the corpus cavernosum and corpus spongiosum. By stimulating guanylate cyclase and increasing the synthesis of cGMP, NO causes a relaxation of smooth muscle cells. Thereby a combination of α₁-antagonistic and α₂-antagonistic effects is regarded as particularly favourable for the treatment of ED because the inhibition of α₁-receptors directly produces a muscle relaxation and the inhibition of presynaptic α₂-receptors is accompanied by an increased release of NO from nitrergic neurons (http://www.bioportfolio.com/leaddiscovery/mdi002.htm).

The treatment of ED has been revolutionized by the development of sildenafil, a PDE5 inhibitor which inhibits the degradation of cGMP. However, sildenafil causes a variety of side effects (such as headaches, impaired vision, dyspepsia, hemodynamic effects). Furthermore, there is evidence that the efficacy decreases with an increasing period of treatment (M. Shabbir et al., Curr. Med. Res. Opin. 20, 603, 2004). Furthermore, sildenafil can not be used by 30-50% of the patients due to the severity of disease and the contraindications. There is currently not a wide clinical experience with new PDE5 inhibitors. Also PGE, is an active substance for the treatment of ED, however, it has to be injected or administered intraurethrally. As a further treatment option apomorphine is available, which acts as a dopamine agonist in the central nervous system. It is suitable for rather less severe cases of ED. Having a generally lower side effect rate (nausea, cardiovascular effects), the sublingual administration which is required to avoid a hepatic first pass metabolization, is regarded as rather disadvantageous (R. B. Moreland et al., J. Pharmacol. Exp. Ther. 2004, 308, 797). Thus, there is still a large demand for effective medicaments for the treatment of ED, which are low in side effects.

In 1999 a publication which shows that the portion of females with sexual dysfunction (about 43%) is significantly higher than that of male (about 31%) in the US (E. O. Laumann et al., JAMA 281, 537, 1999), has been recognized among experts with surprise. In general, sexual dysfunctions in female are classified into four categories: lack of sexual desire or sexual aversion, reduced sexual excitability, painful sexual intercourse (vaginism, dyspareunia) and orgasmic disorders. The portion of these different disorders are 30% (lack of sexual desire), 20% (reduced sexual excitability), 10 to 15% (painful sexual intercourse) and 10 to 15% (orgasmic disorders), wherein there is a very close relationship between the different kinds of sexual dysfunctions.

The regular sexual function in male and female is controlled via a response cycle which consists of a mental expectation (sexual desire), effective vasocongestion (erection in men, swelling of the clitoris and vaginal lubrication in women), orgasm and finally resolution. This overall course of events depends on a balanced equilibrium between the parasympathetic and sympathetic nervous system. Thereby, the vasocongestion of the sexual organs is of central importance. Since there is an analogy between the penis and the clitoris with respect to the anatomic structure and the innervation of the corpora cavernosa, it is to be anticipated that the same pharmacological mechanisms, which are efficient in the treatment of erectile dysfunction, can also be employed in the treatment of female sexual disorders and particularly reduced sexual irritability.

Therefore, it is the object underlying the present invention to provide medicaments which positively effect both the dynamic and static component of BPH by inhibiting α₁-adrenoceptors and α₂-adrenoceptors and by inhibiting the growth factor mediated proliferation of epithelial cells and stromal cells and, thus, enable the comprehensive treatment of the BPH syndrome, LUTS, ED and other sexual dysfunctions in men and women as well as hypercholesterolemia, functional disorders of the bladder, cardiovascular diseases-and-pain-conditions.

According to the present invention, this object is solved by the use of extracts from the bark of corynanthe species, preferably corynanthe pachyceras for the therapy and prophylaxis of diseases of the lower urinary tract in men and women (for example benign prostatic hyperplasia, LUTS, carcinoma of the prostate, disorders in emptying the bladder, urinary retention, stress incontinence and urge incontinence), sexual disorders in men and women (such as impotence, erectile dysfunction, premature ejaculation, libido disorders, frigidity or anorgasmy), disorders of the lipid metabolism (for example hypercholesterolemia, hyperlipidemia, hypertriglycerinemia), cardiovascular diseases (for example endothelial dysfunction, hypertonia, arteriosclerosis or restenosis after vasodilatation or bypass surgery) and acute and chronic pain conditions such as migraine, neuropathic pains (for example in case of diabetes), phantom limb pain, allodynia, pains after tissue injuries or in case of inflammations (for example postherpetic neuralgia).

Further subjects of the present invention are extracts from the bark of corynanthe species, preferably from the bark of corynanthe pachyceras, which have a balanced ratio of effective components, as well as medicaments and food products for the therapy and prophylaxis of diseases of the lower urinary tract, sexual disorders, disorders of the lipid metabolism, cardiovascular diseases and acute and chronic pain conditions, which are characterized by a content of an extract according to the present invention, as well as a pharmaceutical preparation as an oral, parenteral or topicalal administration form. As used herein, the term “food products” particularly refers to dietetic food products, dietary supplement products as well as medical food and dietary supplements.

Corynanthe pachyceras (rubiaceae) is a tree having a height of 15-20 m and a trunk diameter of up to 60 cm in the evergreen tropical rain forest in Western Africa (Sierra Leone to Zaire). The wood is prevalently used for building purposes, but also for the manufacture of mortars and combs. The dried bark of the trunk is extensively used in traditional medicine. The bark is chewed against colds and used as a decoction in case of leprosy, gastric complaints, diarrhea or cardiac and renal complaints. The bark is used in the form of teas as an antipyretic agent in case of malaria and as an aphrodisiac and a wake-promoting agent.

The bark of corynanthe pachyceras contains about 6% of indolalkaloids which are assigned to the group of corynantheine alkaloids (for example dihydrocorynanteine, corynantheine, corynantheidine) or yohimbine alkaloids (for example corynanthine, α-yohimbine). For the particular corynanthe alkaloids the focus lies on their α-adrenoceptor antagonistic activity. Furthermore, a leishmanicidal activity at a moderate cytotoxicity for mammal cells and plasmodium falciparum is described (D. Staerk et al., Planta Med. 2000, 66, 531, 2000).

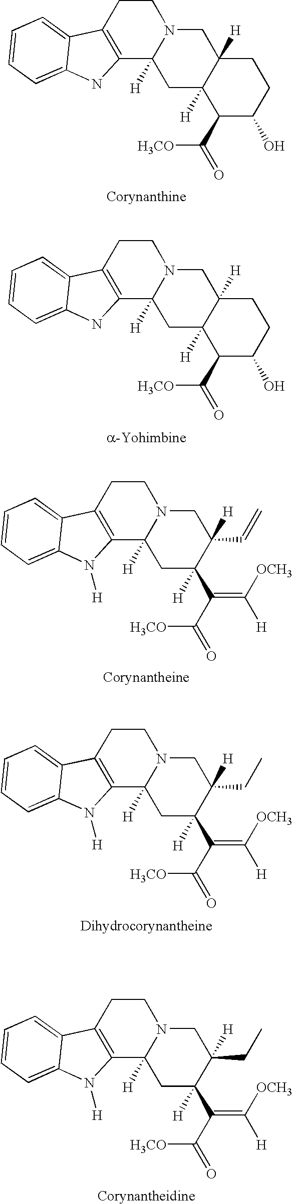

A patent specification from the year 1971 claims antihypertensive and sedative effects of an aqueous dry extract from the bark of corynanthe pachyceras (BE758049, Omnium Chimique SA, 1971).

We now have surprisingly observed that alcoholic or ketonic, preferably ethanolic-aqueous extracts from the bark of corynanthe species, in particular corynanthe pachyceras exhibit a variety of further biological effects such as cell proliferation inhibiting properties, endothelium dependent vasorelexing properties, cholesterol lowering properties, analgetic and antioxidative properties, in addition to α₁-adrenoceptor antagonistic and α₂-adrenoceptor antagonistic effects. These different activities suggest the therapeutic use of these extracts against various disease conditions. BPH, LUTS, sexual dysfunction in men and women, functional disorders of the bladder, hypercholesterolemia, arteriosclerosis, endothelial dysfunctions and pain conditions are among this diseases. The efficacy of the extracts according to the present invention against these indications is supported by the following pharmacologic investigations. In these investigations it has turned out to be essential for the effect of the extracts according to the present invention that the extracts have to contain polyphenols as active ingredients in addition to alkaloids. Thereby, the term “polyphenols” refers to aromatic compounds having at least two hydroxyl groups which may be present in the monomeric, oligomeric or polymeric form. Extracts containing both groups of compounds are clearly superior to the isolated ingredients from corynanthe pachyceras with respect to their overall effect.

The extracts according to the present invention from the bark of corynanthe species, which have a content of polyphenols and alkaloids, can be obtained according to the following method:
(a) extracting dried and ground bark of corynanthe species with an organic solvent or water or a mixture of one or more organic solvents and/or water at a temperature between 10° C. and 100° C.,
(b) separating the extracted plant material from the extract solution, for example by filtration,
(c) optionally reextracting the extracted plant material with a solvent according to step (a) and separation according to step (b),
(d) combining the extract solutions obtained in steps (b) and (c),
(e) evaporating and drying the combined solution from step (d) to yield the dry extract.

Preferred organic solvents in step (a) are alcohols or ketones, wherein the alcohol is preferably ethanol. Mixtures of ethanol and water are particularly preferred. Maceration and percolation may be preferably taken into consideration as the extraction method in step (a). As a general rule, step (c) is carried out once and waving step (c) or a plural performance is possible as well. The drying in step (e) can be carried out by methods known per se, such as lyophilization or drying in vacuum at room temperature or elevated temperature.

Corynanthe pachyceras is employed as the preferred corynanthe species.

The extracts according to the present invention from the bark of corynanthe species contain both polyphenols and alkaloids in a ratio balanced for the intended use. Thereby, the content of polyphenols is preferably at least 15%, particularly preferred at least 24% and the content of alkaloids is preferably at least 8%, particularly preferred at least 12%. The compounds epicatechine, procyanidin B2 and procyanidin C1 have been isolated by us from corynanthe pachyceras as typical polyphenols. However, corynanthe polyphenols are not limited to these three compounds.

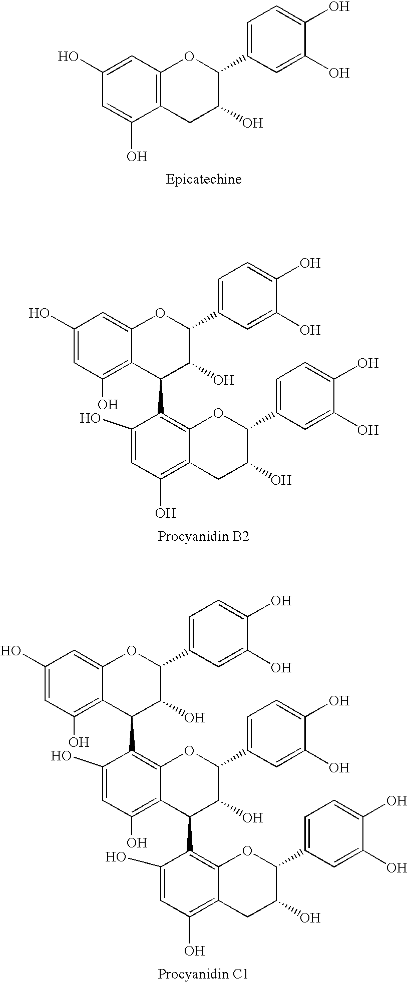

A determination of the contents of polyphenols was carried out by determining the total phenol content according to Folin-Ciocalteu. Additionally or alternatively, the contents of epicatechine, procyanidin B2 and procyanidin C1 can also be determined. The alkaloid contents mentioned are the sum of the contents of the individual alkaloids corynanthine, α-yohimbine, corynantheine, dihydrocorynantheine and corynantheidine.

The extracts and extract fractions according to the present invention may be administered in the form of droplets, powders, granules, tablets, coated tablets (dragees) or capsules, preferably orally. However, a parenteral application in the form of an injection solution or a topicalal application in the form of crimes, ointments, suppositories, patches or similar preparations is also possible.

For the preparation of tablets the extract is mixed with suitable pharmaceutically acceptable adjuvants such as lactose, cellulose, silicon dioxide, croscarmellose and magnesium stearate and pressed into tablets which may optionally be provided with a suitable coating made of, for example, hydroxymethylpropylcellulose, polyethyleneglycol, colorants (such as titanium dioxide, iron oxide) and talcum.

The extracts according to the present invention may also be filled into capsules optionally under the addition of adjuvants such as stabilizers, fillers and the like.

The dosage is such that 5 to 2000 mg, preferably 10 to 1000 mg and particularly preferred 50 to 500 mg extract are administered per day.

The efficacy of the extracts according to the present invention from the bark of corynanthe species is supported by the experiments described in the following.

### Pharmacological Investigations

Assay for α-adrenoceptor Binding Properties

The test of corynanthe extracts and fractions of corynanthe extracts for interactions with α-adrenoceptors was carried out by a receptor binding assay using brain cell membranes of rats. For the preparation of cell membranes, male Sprague-Dawley rats (150-250 g) were euthanized in CO₂ narcosis and the brains were removed (without the cerebellum). After the removal of adhering blood and meninges, the brains were immediately taken up in ten times their volume made up of ice cold homogenization buffer (50 mM Tris-HCl, pH 7.4) and homogenized using an ice cooled glass homogenizor. The cell homogenate was centrifugated for 10 minutes at 50,000 g (4° C.) and the pellet was resuspended in ice cold homogenization buffer. After a further centrifugation (10 min at 4° C. and 50,000 g) the cell membranes were taken up in ten times their volume made up of binding buffer (50 mM Tris-HCl, 0.5 mM Na-EDTA, 0.01% ascorbic acid, 10 μM pargylin, pH 7.4) and stored in portions (1 ml) at −80° C.

The extract according to the present invention or the alkaloid fractions or the polyphenol fractions were dissolved using DMSO in 150 μl binding buffer and incubated together with 50 μl brain cell membranes (2.5 mg/ml protein) and 50 μl radioactive ligand in binding buffer for 45 minutes at room temperature. ³H-prazosin (300 pM, specific activity 80 Ci/mmol) was used as the radioligand for the assay regarding interactions with α₁-adrenoceptors. The unspecific binding was measured in the presence of 2 μM phentolamine. ³H-clonidine (1 μM, specific activity 55.5 Ci/mmol) served as the radioligand for the determination of the α₂-adrenoceptor binding. The assay for the unspecific binding to α₂-adrenoceptors was carried out in the presence of 10 μM yohimbine. The reaction mixtures were subsequently filtered through glass fiber filters (type GF/B) which had been pre-treated with polyethylene imine (0.2% in aqua dest.) overnight. After washing the filters twice using 3 ml ice cold binding buffer each time, the filters were dried for 24 hours at 60° C. The determination of the bound radioactivity was carried out after transferring the filters into 4 ml scintillization liquid (filter safe, Zinsser-Analytik) in a beta counter. The percentage of inhibition of the specific binding of ³H-prazosin to α₁-adrenoceptors or ³H-clonidine to α₂-adrenoceptors was calculated as compared to the solvent control which had been investigated simultaneously. The determination of the half maximum inhibition concentration (IC₅₀ values) was performed by non-linear regression calculation.

The results of the investigations are summarized in Table 1. It can be seen that the extract according to the present invention from the bark of corynanthe inhibits both the binding of ³H-prazosin to α₁-adrenoceptors and the binding of ³H-clonidine to α₂-adrenoceptors, this effect being essentially based on the presence of alkaloids.

TABLE 1
Inhibition of the binding of ³H-prazosin and ³H-clonidine
to α₁-adrenoceptors and α₂-adrenoceptors, respectively

| | Inhibition of the receptor binding of | |
|---|---|---|
| | ³H-prazosin or ³H-clonidine (half | |
| | maximum inhibition concentration, IC₅₀) | |
| | α₁-receptor | α₂-receptor |
| | binding | binding |
| Extract according to | 0.8 μg/ml | 2.1 μg/ml |
| the present invention | | |
| (Example 1) | | |
| Polyphenol fraction | 10 μg/ml | 10 μg/ml |
| (Comparative Example 1) | no inhibition | no inhibition |
| Alkaloid fraction | 0.05 μg/ml | 1 μg/ml |
| (Comparative Example 2) | | |

### Assay for the Inhibition of Growth Factor Mediated Cell Proliferation

The influence of the overall extracts or the extract fractions on the growth factor induced cell proliferation was assayed on NIH-3T3 fibroblasts of mice. The cells were cultivated in Dulbecco's modified Eagle's medium (DMEM) supplemented with 10% fetal calf serum (FCS), 2 mM glutamine and antibiotic/antimycotic solution. The culture medium was regularly replaced twice a week. Four days after the last subcultivation, adherent cells were detached using trypsine/EDTA from the bottom of the cell culture bottle and resuspended in a density of 50,000 cells per ml in DMEM supplemented with 0.5% FCS. Subsequently, the cells were transferred in a volume of 200 μl per well into microtiter plates (F-form) and incubated at 37° C. for further 96 h. After replacing the medium (DMEM without FCS) and adding the substances, 10 ng/ml recombinant human platelet-derived growth factor BB (PDGF-BB) was added 60 minutes later. Subsequently, the cells were cultivated again for 24 h at 37° C. in an incubator. Six hours prior to harvesting the cells, 0.5 μCi methyl-3H-thymidine was added per well. After the expiry of the incubation period the microtiter plates were centrifuged for 5 min at 400 g and the cell supernatants were carefully pipetted off. The cells were detached from the bottom using trypsine/EDTA and subsequently harvested on glass fibre filters (type G-10, ICH-201) using a cell harvester (Inotech). The determination of the incorporation of ³H-thymidine in newly synthesized DNA was carried out by means of a linear analyzer (LB 2842, Berthold). The determination of the inhibition of the cell proliferation in the presence of the extracts and the extract fractions was carried out in comparison to simultaneously assayed solvent controls in each case.

A summary of the results is given in Table 2. It can be seen from these results that the cell proliferation inhibiting action of the extracts according to the present invention can mainly be attributed to the polyphenol fraction.

TABLE 2
Inhibition of the PDGF-mediated cell
proliferation of NIH3T3 fibroblasts

| | half maximum inhibition concentration |
|---|---|
| Extract according to the | 5.4 μg/ml |
| present invention | |
| (Example 1) | |
| Polyphenol fraction | 3.0 μg/ml |
| (Comparative Example 1) | |
| Alkaloid fraction | 12.3 μg/ml |
| (Comparative Example 2) | |

### Assay for Vasorelaxing Properties

For assaying the vasorelaxing effects the influence of extracts according to the present invention and extract fractions on the contraction of the isolated aorta of male Sprague-Dawley rats (Janvier, Le Genest, France) was examined. Immediately after removing, the organs were transferred into Tyrode's solution (in mM: NaCl 118.2, NaHCO₃ 24.8, KCl 4.6, CaCl₂ 2.5, MgSO₄ 1.2, KH₂PO₄ 1.2, glucose 10) and set free from adhering connective tissue. Then vascular rings having a thickness of about 4 mm were prepared. For some experiments the endothelium was removed. For this purpose the aorta rings were mounted on a steel canula, slightly pressed against the steel canula and the innermost vascular layer was subsequently removed by turning and simultaneously moving in longitudinal direction. The aorta rings were fixed in an organ bath (20 ml; Hugo Sachs, Hugstetten) filled with Tyrode's solution using metal hooks. The culture medium (37° C.) was permanently gassed with carbogen (pH 7.4). For experiments regarding the determination of the vascular relaxation after a precontraction of the organs using phenylephrine (PE), the following substances were added to the Tyrode's solution: propanolol-HCl (6 μM; RBI), corticosterone-HCl (6 μL; Sigma) and desipramine (0.6 μM; Sigma). In tests regarding the relaxation after stimulation using U-46619 indomethacin (2.8 μM; Sigma) was added. The organ tension was measured isometrically using a force transducer (Statham UC2, Hugo Sachs) under a preload of 1.0 g and recorded using a 4-channel-writer (Linearcorder, Graphtec). After an equilibration phase of 30 min four contractions were elicited with PE (0.15 iμg/ml, EK 0.74 μM) in intervals of 15 minutes in order to achieve reproducible organ contractions. Following the fourth PE addition, the test substance was added in an increasing concentration after reaching the contraction maximum (cumulative dosage effect). At the end of the experiment the endothelium dependency of the vasorelaxation was tested by the application of acetylcholine (0.25 μg/ml, EK 1.38 μM).

A basically similar test procedure was complied with also for testing the relaxing effect on aorta rings without endothelium. After equilibration for 30 minutes, three contractions were elicited with PE (0.15 μg/l, EK 0.74 μM) in intervals of 15 minutes. After reaching the contraction maximum subsequent to the third addition of PE, acetylcholine was applied (0.25 μg/ml, EK 1.38 μM) in order to confirm the complete removal of the endothelium. After rinsing out the acetylcholine, a PE contraction (0.15 μg/ml, EK 0.74 μM) was elicited after 25 minutes and subsequently the test substance was added in increasing concentrations.

As described above with respect to PE, assays for a relaxing effect after inducing a contraction with U46619 (0.022 μg/ml, EK 0.063 μM) or KCl (3 mg/ml, EK 40 mM) was carried out in an identical manner. The relaxing effect of the extracts on the agonists was determined as a percentage. IC₅₀ values were determined by a non-linear regression analysis of concentration-effect-curves using the Prism 3.0 software (GraphPad Software Inc.).

The results of the experiments are shown in the following table. It is evident from the data that the vasorelaxing effect of the extract according to the present invention after stimulation with PE is mediated by both the alkaloid containing fraction and the alkaloid free fraction. The relaxing effect of the alkaloid free fraction is completely dependent on the presence of an intact endothelium. The endothelium-dependent vasorelaxing effects of the alkaloid free fraction could also be observed after precontraction of the vascular rings with U-46619 or KCl-depolarization and are obviously based on the increased endothelial release of NO. In contrast, the effect of the alkaloid fraction is mainly based on the presence of ingredients having α-adrenoceptor blocking properties and is, thus, also determinable after removing the endothelium.

TABLE 3
Assay for vasorelaxing effects

| | vaso- | vaso- | vaso- | vaso- |
|---|---|---|---|---|
| | relaxation, | relaxation, | relaxation, | relaxation, |
| | PE- | PE- | U-46619- | KCl- |
| | contraction, | contraction, | contraction, | contraction, |
| | endothelium | endothelium | endothelium | endothelium |
| | intact | removed | intact | intact |
| Extract according to the | ED50: | ED50: | 25 μg/ml: | 25 μg/ml: |
| present invention | 0.7 μg/ml | 6.3 μg/ml | 74% | 61% |
| (Example 1) | | | | |
| Polyphenol fraction | ED50: | 25 μg/ml: | ED50: | 25 μg/ml: |
| (Comparative Example 1) | 16 μg/ml | inactive | 16 μg/ml | 25% |
| Alkaloid fraction | ED50: | ED50: | 25 μg/ml: | 25 μg/ml: |
| (Comparative Example 2) | 0.22 μg/ml | 0.23 μg/ml | 15% | inactive |

### Assay on Anti-Hypercholesterolaemic Effects

The influence of the extract according to the present invention on the plasma cholesterol level was examined on male NMRI mice (Janvier, Le Genest, France) with Triton WE1339-induced hypercholesterolaemia. The mice were kept under standardized environmental conditions (21° C., 60% relative humidity, 12/12 h bright/dark) and had free access to drinking water and pelletized feed (Altromin 1324). Triton-WR1339 (400 mg/kg, Sigma) was dissolved in physiological NaCl solution and injected into the animals via the caudal vein (10 ml/kg). For an oral treatment of the test animals, the extract was taken up in 0.2% agar suspension and administered to the animals 24 h and 1 h prior to the injection of Triton-WR1339 as well as 6 h after the injection of Triton-WR1339 via gavage (450 mg/kg in 10 ml/kg). Animals in the control group were treated with the carrier (0.2% agar, 10 ml/kg) only. One hour prior to the injection of Triton-WR1339 and 6 h, 24 h and 48 h after the injection of Triton-WR1339, a blood sample (32 μl) was taken from the caudal vein of the animals using a heparinized capillary and the cholesterol concentration was determined immediately thereafter (Reflotron, Boehringer Mannheim).

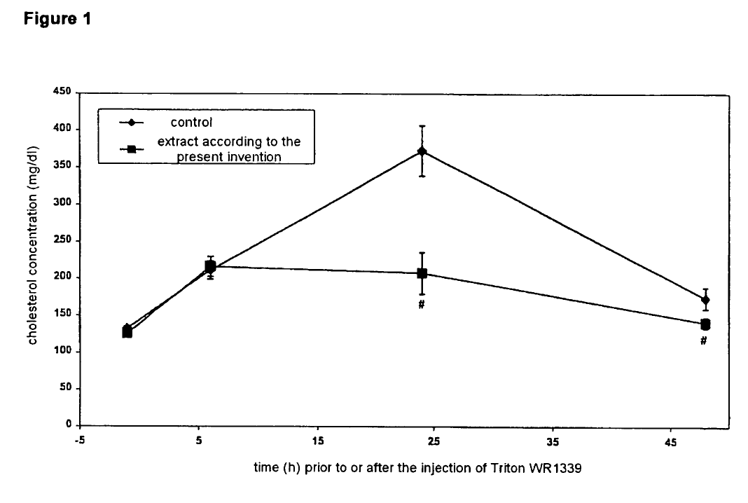

FIG. 1 shows the influence of the oral treatment with the extract according to the present invention (450 mg/kg) on the cholesterol level in mice with Triton WR1339-induced hypercholesterolaemia (# means P<0.05 as compared to the control (t-test)). The results of the experiments demonstrate that the treatment with the extract according to the present invention obtained in Example 1 leads to a significant reduction of the increased cholesterol level.

### Assay for Analgetic Effects

For assaying analgetic effects the formalin test was used in mice. The local injection of formalin into the hind foot of mice leads to an increased sensitivity to pain which occurs in two temporally separated phases. The first phase is mediated by a direct stimulation of the pain receptors due to the release of substance P, bradykinin and excitatory amino acids (e.g. glutamine). In the subsequent second phase, an accumulation of histamine, serotonine and prostaglandins in the tissue occurs, which leads to a local inflammation reaction and functional changes in the central nervous system. For the experiments, male NMRI mice (Janvier, Le Genest, France) having a weight of about 22-26 g were used. The animals were treated orally with the extracts according to the present invention or extract fractions. One hour later, 20 μl of a 3.5% formalin solution was injected into the left sole of foot. Subsequently, the animals were individually placed into wire cages and the number of pain reactions (licking the paw) was recorded over a period of 45 min. The pain inhibiting effect was determined as compared to a simultaneously tested control group. The animals in this group were treated with a carrier medium only (0.2% agar suspension, 10 ml/kg).

The results in Table 4 demonstrate the potent analgetic effects of corynanthe extracts which are substantially mediated by the alkaloids contained therein.

TABLE 4
Assay for analgetic effects

| | inhibition of the pain reactions |
|---|---|
| Extract according to the | 450 mg/kg |
| present invention | −76% |
| (Example 1) | |
| Polyphenol fraction | 450 mg/kg |
| (Comparative Example 1) | −22% |
| Alkaloid fraction | 100 mg/kg |
| (Comparative Example 2) | −76% |

### Assay for Antioxidative Properties

The autoxidation of lipids is associated with the emission of light. The determination of this extraordinarily weak chemiluminescence can be used both for quantifying peroxides and for assessing the efficacy of antioxidants. Brain tissue of male mice (NMRI; 20-30 g; Centre d'Elevage Janvier, Le Genest-Saint Isle, France) served as lipid-rich tissue in the present investigations. After its removal the brain was washed with ice cold phosphate-buffered physiological saline (PBS, pH 7.4) and set free from meninges and remaining blood. The tissue samples were homogenized in 4 times their volume (v/w) made up of PBS and centrifuged for 10 minutes at 1000 g and 4° C. The supernatants were immediately diluted to three times their volume with the same buffer and stored on ice. 250 μl of the diluted supernatant was transferred into a test tube and incubated for 10 minutes at 37° C. in a 6-channel luminometer (Multi-Biolumat LB 9505 C, Berthold, Bad Wildbad). After adding 25 μl of compound II in PBS with 2.5% DMSO, the incubation was continued for a further 10 minutes. Then the intensity of the chemiluminescence (CL) was determined for a period of 60 minutes. The percentage of inhibition of the autoxidation was calculated as compared to a simultaneously tested solvent control (PBS with 2.5% DMSO). As can be taken from the results summarized in Table 5, the extract according to the present invention exhibits potent antioxidative properties which are primarily mediated by the polyphenol fraction.

TABLE 5
Assay for antioxidative properties

| | inhibition of the lipid |
|---|---|
| | peroxidation IC₅₀ (mg/ml) |
| Extract according to the | 1151 |
| present invention | |
| (Example 1) | |
| Polyphenol fraction | 916 |
| (Comparative Example 1) | |
| Alkaloid fraction | 4786 |
| (Comparative Example 2) | |

### EXAMPLES

### Determination of the Total Content of Phenols According to Folin-Ciocalteu

The determination of the total content of phenols is carried out photometrically after reacting with molybdate-wolframate-reagent in analogy to the pharmacopoeia method for tanning agents (DAB). For this purpose the extract is dissolved in aqueous ethanol, alkalized with sodium carbonate solution and added with molybdate-wolframate-reagent. After centrifugation the absorbance of the supernatant solution is measured against water at 720 nm. The calculation is based on epicatechine.

### Example 1

### Dry Extract According to the Present Invention from the Bark of Corynanthe Pachyceras

500 g of ground bark of corynanthe pachyceras was stirred twice for one hour at 60° C. using 3.5 kg of 60% by weight ethanol each time. After filtration over Seitz Supra 1500, the combined extract solution were evaporated at about 50° C. and reduced pressure and dried at 50° C. in vacuum: 183.8 g (36.8%).

The extract contains 14.62% alkaloids (4.75% corynanthine, 0.81% α-yohimbine, 3.86% corynantheine, 1.91% dihydrocorynantheine and 3.29% corynantheidine) and had a total phenol content of 26.8% (including 2.66% epicatechine, 3.05% procyanidin B2 and 1.25% procyanidin C1).

### Comparative Example 1

### Polyphenol Fraction (Alkaloid-Free)

A solution of 445 g dry extract according to Example 1 in 4 kg ethanol (50% by volume) is placed on a column with 3.4 L strongly acidic ion exchanger (Merck I) and eluted with ethanol (50% by volume). 9 L eluate were collected, evaporated at 50° C. under reduced pressure and dried in a drying cabinet at 50° C. and 12 mbar: 352.4 g (79.2%).

The extract does not contain any alkaloids (corynanthine, α-yohimbine, corynantheine, dihydrocorynantheine and corynantheidine could not be detected) and exhibited a total phenol content of 28.4% (including 2.57% epicatechine, 2.24% procyanidin B2 and 0.76% procyanidin C1).

### Comparative Example 2

### Alkaloid Fraction

The ion exchanger column from Comparative Example 1 was further eluted using a mixture of 50% by volume ethanol and 5% NH₃ solution (having a concentration of 25%). 16 L eluate were collected and evaporated and dried as in Example 2: 46.8 g (10.5%).

The extract contains 69.28% alkaloids (24.01% corynanthine, 2.25% α-yohimbine, 19.05% corynantheine, 9.56% dihydrochorynanteine and 14.41% corynantheidine) and has a total phenol content of 13.0% (epicatechine, procyanidin B2 and procyanidin C1 could not be detected).

### Example 2

### Tablets

A dry extract form the bark of corynanthe pachyceras (extract according to the present invention obtained in Example 1) is mixed with adjuvants and pressed into tables (tablet core=items 1-6). The tablets are provided with a coating made of hydroxypropyl methyl cellulose (items 7-10).

| | ingredient | mg/tablet |
|---|---|---|
| 1 | dry extract from the bark of | 100.0 |
| | corynanthe pachyceras (Example 1) | |
| 2 | microcrystalline cellulose | 117.0 |
| 3 | lactose-monohydrate | 58.0 |
| 4 | croscarmellose | 15.0 |
| 5 | highly dispersed silicon dioxide | 3.0 |
| 6 | magnesium stearate | 6.0 |
| 7 | hydroxypropylmethyl cellulose | 15.0 |
| 8 | polyethylene glycol | 3.0 |
| 9 | talcum | 1.0 |
| 10 | titanium dioxide | 2.0 |

1-12. (canceled)

13. An extract from the bark of corynanthe pachyceras having a content of polyphenols and alkaloids, the extract obtainable from a process comprising steps of:
(a) extracting dried and ground bark of corynanthe pachyceras with an organic solvent or a mixture of several organic solvents or a mixture of one or more organic solvents and water at a temperature between 10° C. and 100° C., wherein the organic solvent is an alcohol or a ketone;
(b) separating the extracted plant material from the extract solution;
(c) optionally reextracting the extracted plant material with a solvent according to step (a) and separating according to step (b),
(d) combining the extract solutions obtained in steps (b) and (c),
(e) evaporating and drying the combined solution from step (d) to obtain the dry extract.

14. An extract of claim 13 wherein the alcohol is ethanol.

15. An extract of claim 13 wherein the extraction solvent is a mixture of ethanol and water.

16. An extract of claim 13 wherein the extract has a content of polyphenols of at least 15 weight % and alkaloids of at least 8 weight %.

17. An extract of claim 16 wherein the extract has a content of polyphenols of at least 24 weight %.

18. An extract of claim 16 wherein the extract has a content of alkaloids of at least 12 weight %.

19. A pharmaceutical composition comprising an extract of claim 13.

20. The pharmaceutical composition of claim 19 further comprising a pharmaceutically acceptable adjuvant.

21. The pharmaceutical composition of claim 19 wherein the pharmaceutical composition is formulated in an oral, parenteral or topical administration form.

22. A method for treating a subject suffering from or susceptible to a disease of the lower urinary tract, sexual disorder, lipid metabolism disorder, cardiovascular disease and/or acute and/or chronic pain condition, the method comprising:
administering an effective amount of an extract of claim 1 to the subject.

21. The method of claim 22 wherein the subject is suffering from or susceptible to impotency, erectile dysfunction, premature ejaculation, frigidity and/or anorgasmy,

22. The method of claim 22 wherein the subject is suffering from or susceptible to endothelial dysfunction, arteriosclerosis, and/or restenosis after vasodilatation or bypass operation.

23. The method of claim 22 wherein the subject is suffering from or susceptible to benign prostatic hyperplasia, LUTS, carcinoma of the prostate, disorder of voiding the bladder, urinary retention, stress incontinence and/or urge incontinence.

24. The method of claim 22 wherein the subject is suffering from or susceptible to hypercholesterolaemia, hyperlipidaemia and/or hypertriglyceridaemia.

25. The method of claim 22 wherein the subject is suffering from or susceptible to migraine, neuropathic pain, phantom limb pain, allodynia, and/or pain after tissue injury and/or in case of inflammation.

26. A food product comprising an extract of claim 13.

27. An extract from the bark of corynanthe pachyceras having a content of polyphenols and alkaloids, the extract obtainable from a process comprising steps of:
(a) extracting dried and ground bark of corynanthe pachyceras with an organic solvent or a mixture of several organic solvents or a mixture of one or more organic solvents and water, wherein one or more of the organic solvents is an alcohol or a ketone; and
(b) separating the extracted plant material from the extract solution.

28. An extract of claim 27 further comprising evaporating and drying the extract composition from step (a) to obtain the dry extract.
